(19) United States
(12) Patent Application Publication
(10) Pub. No.: US 2009/0098221 A1
(43) Pub. Date: Apr. 16, 2009

(54) Creatine ascorbyl derivatives and methods of use thereof

(76) Inventor: Belinda Tsao Nivaggioli, Atherton, CA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/151,285

(22) Filed: May 5, 2008

Related U.S. Application Data
(60) Provisional application No. 60/927,468, filed on May 3, 2007.

(51) Int. Cl. A61K 36/00; C07F 9/09; A61P 25/00; A61P 29/00; A61P 17/00; A61K 31/665

(52) U.S. Cl. 424/725; 549/222; 514/99

(57) ABSTRACT

The present invention provides methods of treating creatine responsive states, such as a neurological disorder (i.e., Huntington's disease, Parkinson's disease, amyotrophic lateral sclerosis, muscular dystrophy, Charcot Marie Tooth syndrome, Alzheimer's disease, or creatine transporter defect) or a skin disorder, by administering a creatine-ascorbyl derivative.

### RELATED APPLICATIONS

This application claims priority to U.S. Provisional Patent Application No. 60/927,468, filed on May 3, 2007. The contents of the foregoing application are hereby incorporated by reference in their entirety.

### BACKGROUND OF THE INVENTION

Creatine is a naturally occurring compound that is found within the mammalian to body, for example, in the brain, heart, retina and skeletal muscle. The lack of creatine in mammalian systems has been implicated in neurological disorders. Neurological disorders are disorders that affect the central nervous system, the peripheral nervous system or the autonomic nervous system. These neurological disorders include, for example, amyotrophic lateral sclerosis (ALS), Huntington's disease, Parkinson's disease and creatine transporter defect. ALS, often referred to as “Lou Gehrig's disease,” is a progressive neurodegenerative disease that attacks the motor neurons of the brain and spinal cord that are responsible for voluntary muscle movement. As these motor neurons degenerate their ability to send impulses to the muscle fibers is compromised. As the disease progresses, the motor neurons die, which results in the brain's inability to initiate or control muscle movement and, eventually, the patient becomes completely paralyzed and their muscles atrophy. ALS affects roughly 30,000 Americans at one time and every year 5600 new cases of ALS are diagnosed. Huntington's disease is a progressive neurodegenerative disease caused by a genetic defect. The disease causes the deterioration of neurons in those parts of the brain that are responsible for controlling cognitive, emotional and motor functions. As a result, patients suffer a variety of symptoms including uncontrollable muscle movements, clumsiness, memory loss, and, ultimately, severe mental deterioration. In the United States, approximately 35,000 people suffer from Huntington's disease and another 175,000 people are at risk for developing the disease. Parkinson's disease is a progressive, neurodegenerative brain disorder that occurs when neurons within the brain that are responsible for producing the chemical dopamine become impaired or die. The cause of this nerve cell damage and death is not completely understood. Eventually, symptoms, which include uncontrolled shaking of the hands and or feet, may progress to a point where routine tasks become severely impaired. It is estimated that approximately 1:5 million Americans are affected by Parkinson's disease, making it the second most common neurodegenerative disease after Alzheimer's disease. Approximately 60,000 new cases are diagnosed each year in the United States. Creatine transporter defect (CTD) is an inherited error of metabolism that inhibits the body's ability to supply sufficient levels of creatine to the brain via the creatine transporter. Caused by a defect in the X-linked creatine transporter, CTD results in mental retardation with symptoms including speech and language impairment, short attention span and low I.Q.

There are currently no known cures for ALS, Huntington's disease, Parkinson's disease, CTD and many other neurological disorders. Instead, treatment is focused on relieving symptoms, preventing complications and maximizing the quality of life.

In addition, the use of creatine and creatine analogues has been shown to be effective for use in the prevention and treatment of skin disorders, such as free-radicals, aging, sun radiation, stress, fatigue, psoriasis, uneven pigmentation or skin damage.

### SUMMARY OF THE INVENTION

In one embodiment, the invention pertains to a creatine composition of the formula (I):

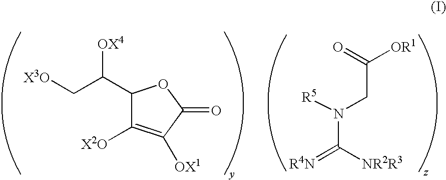

wherein:

X¹, X², X³, and X⁴ are each independently hydrogen, phosphate, diphosphate, triphosphate, sulfate, or carboxylate, provided that at least one of X¹, X², X³, and X⁴ is not hydrogen;

R¹ is hydrogen, phosphate, diphosphate, triphosphate, sulfate, or carboxylate;

R², R³, R⁴ and R⁵ are each independently alkyl or hydrogen; and

x and y are each independently selected integers, or a pharmaceutically acceptable salt or tautomer thereof.

In another embodiment, the invention also pertains, at least in part, to pharmaceutical compositions comprising a pharmaceutically acceptable carrier and a creatine composition of the formula (I).

In another embodiment, the invention also includes a method of treating a creatine responsive state in a subject, by administering to the subject a composition comprising an effective amount of a creatine composition of formula (I), such that the creatine responsive state in the subject is treated.

In yet another embodiment, the invention also features a method for treatment of a skin disorder. The method includes administering an effective amount of a creatine composition of formula (I) to a subject, such that the skin disorder in the subject is treated.

### DETAILED DESCRIPTION OF THE INVENTION

### Creatine Compositions

The present invention pertains, at least in part, to creatine compositions of the formula (I):

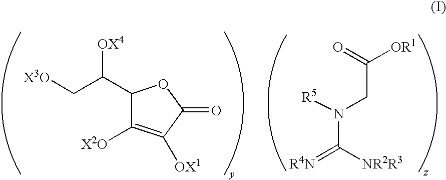

wherein:

X¹, X², X³, and X⁴ are each independently hydrogen, phosphate, diphosphate, triphosphate, sulfate, or carboxylate, provided that at least one of X¹, X², X³, and X⁴ is not hydrogen;

R¹ is hydrogen, phosphate, diphosphate, triphosphate, sulfate, or carboxylate;

R², R³, R⁴ and R⁵ are each independently alkyl or hydrogen; and

x and y are each independently selected integers, or a pharmaceutically acceptable salt or tautomer thereof.

In a further embodiment, each of R¹, R², R³, and R⁴ are hydrogen. In another further embodiment, R⁵ is methyl.

In another further embodiment, at least one of X¹, X¹, X³, or X⁴ is phosphate. For example, X¹ may be phosphate and each of X², X³, and X⁴ may each be hydrogen.

In another embodiment, z is greater than or equal to y. In a further embodiment, the creatine composition of formula (I) is creatine ascorbyl phosphate or a compound of

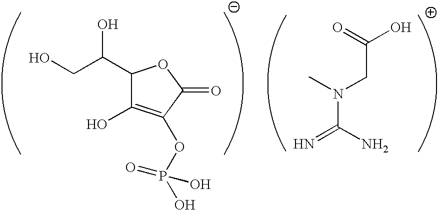

In another embodiment, the present invention also pertains, at least in part to a composition comprising a creatine composition of formula (I) and dextrose (also known as α-D-glucose).

The ratio of z to y can be, for example, about a 1:1 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio or about a 10:1 ratio. The ratio of y to z can also be any ratio in which creatine is bound to the ascorbyl group through covalent or electrostatic interactions. In one embodiment, the creatine composition of formula (I) has a ratio of z:y between about 3:1 z:y and about z:y creatine to ascorbyl phosphate.

In one embodiment, the amount of the creatine composition of formula (I) in the composition is between about 1 gram and about 50 grams. The term “about,” as used with reference to an amount of the creatine composition of formula (I) and/or a second agent and/or dextrose refers to ±0.5 grams of creatine composition of formula (I) and/or a second agent and/or dextrose. In another embodiment, the amount of the creatine composition of formula (I) in the composition is about 1 gram, about 2 grams, about 3 grams, about 4 grams, about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams, about 20 grams, about 21 grams, about 22 grams, about 23 grams, about 24 grams, about 25 grams, about 26 grams, about 27 grams, about 28 grams, about 29 grams, about 30 grams, about 31 grams, about 32 grams, about 33 grams, about 34 grams, about 35 grams, about 36 grams, about 37 grams, about 38 grams, about 39 grams, about 40 grams, about 41 grams, about 42 grams, about 43 grams, about 44 grams, about 45 grams, about 46 grams, about 47 grams, about 48 grams, about 49 grams or about 50 grams or greater. In another embodiment, the amount of the creatine composition of formula (I) is a therapeutically effective amount.

In one embodiment, the amount of dextrose in the composition is between about 1 gram and about 20 grams. In another embodiment, the amount of dextrose in the composition is about 1 gram, about 2 grams, about 3 grams, about 4 grams, about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams and about 20 grams. In another embodiment, the amount of dextrose is necessary to enhance the flow characteristics of the composition.

The creatine composition of formula (I) may also be mixed with any appropriate carrier. Suitable carriers include any pharmaceutically acceptable carrier (e.g., dextrose). An appropriate carrier can be selected such that it blends well with the creatine composition of formula (I) (e.g., similar size, consistency or color). The carrier may also be chosen to enhance the flow characteristics of the creatine composition of formula (I).

In one embodiment, the creatine composition of formula (I) is a pharmaceutically acceptable salt, such as that of magnesium. The compositions of the invention are capable of forming a wide variety of base salts. The chemical bases that may be used as reagents to prepare pharmaceutically acceptable base salts of those compositions of the invention that are acidic in nature are those that form non-toxic base salts with such compositions. Such non-toxic base salts include, but are not limited to those derived from such pharmaceutically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. The pharmaceutically acceptable base addition salts of the compositions of the invention that are acidic in nature may be formed with pharmaceutically acceptable cations by conventional methods. Thus, these salts may be readily prepared by treating the composition of the invention with an aqueous solution of the desired pharmaceutically acceptable cation and evaporating the resulting solution to dryness. In a Finer embodiment, the salt of the creatine composition of the invention is a magnesium salt.

The compositions of the invention may further comprise bivalent metal ions, such as chromium, selenium, zinc, magnesium, iron or calcium. Other bivalent metal ions are also included in the compositions of the invention.

### Methods of Treating Creatine Responsive States

In another embodiment, the invention pertains to a method of treating a creatine responsive state in a subject comprising administering to said subject an effective amount of a creatine composition of formula (I) such that the creatine responsive state in said subject is treated.

As used herein, the term “creatine responsive state” refers to states which can be treated, prevented or otherwise ameliorated by the administration of a creatine composition of formula (I) of the invention. The language “treating a creatine responsive state” is intended to include prevention of the state, amelioration and/or arrest of a preexisting state, and the elimination of a preexisting state. In one embodiment, the creatine responsive state is a neurological disorder. In another embodiment, the creatine responsive state is a skin disorder.

1. Neurological Disorders

In one embodiment, the creatine responsive state is a neurological disorder. In another embodiment, the subject is at risk of suffering from a neurological disorder (e.g., ALS, creatine transporter defect, Huntington's disease, Alzheimer's disease, Charcot Marie Tooth syndrome, muscular dystrophy, Parkinson's disease, etc.). The term “neurological disorder” refers to disorders that may cause a disturbance in the structure or function of the nervous system resulting from developmental abnormalities, disease, genetic defects, injury or toxin. These disorders may affect the central nervous system (e.g., the brain, brainstem and cerebellum), the peripheral nervous system (e.g., the cranial nerves, spinal nerves, and sympathetic and parasympathetic nervous systems) and/or the autonomic nervous system (e.g., the part of the nervous system that regulates involuntary action and that is divided into the sympathetic and parasympathetic nervous systems). Examples of neurological disorders may include, for example, Alzheimer's disease, Landau-Bluffer syndrome, acquired epileptiform aphasia, acute disseminated encephalomyelitis, adrenoleukodystrophy, neurological complications of acquired immunodeficiency syndrome (AIDS), Alexander disease, Alper's disease, amyotrophic lateral sclerosis, ataxia, ataxia-telangiectasia, dysautonomia, autonomic dysfunction, familial dysautonomia, Riley-Day syndrome, benign essential blepharospasm, blepharospasm, monomelic amyotrophy, benign focal amyotrophy, Hirayama syndrome, O'Sullivan-McLeod syndrome, subcortical arteriosclerotic encephalopathy, traumatic brain injury, Brown-Sequard syndrome, Kennedy's disease, bulbospinal muscular atrophy, spinal muscular atrophy, Caravan disease, leukodystrophy, central cord syndrome, cerebellar degeneration, cerebral atrophy, Charcot-Marie-Tooth disease, chorea, dyskinesia, Syndenham chorea, neuroacanthcytosis, Levine-Critchley syndrome, choreoacanthocytosis, chronic inflammatory demyelinating polyneuropathy (CIDP), congenital myasthenia, congenital myopathy, central core disease, nemaline rod myopathy, centronuclear (myotubular) myopathy, corticobasal degeneration, Creutzfeldt-Jakob disease, dementia, dyssenergia cerebellaris myoclonica, dyssenergia cerebellaris progressive, dentate cerebellar ataxia, dentatorubral atrophy, primary dentatum atrophy, Ramsey-Hunt syndrome, dermatomytosis, Devic's syndrome, Schilder's disease, myelinoclastic diffuse sclerosis, dystonias, familial periodic paralysis, Friedreich's ataxia, Germann-Straussler-Scheinker disease, Krabbe disease, Guillain-Barre syndrome, hemiplegia alterans, tropical spastic paraparesis, retrovirus-associated myelopathy, HTLV-1 associated myelopathy, Huntington's disease, hypeitonia, Isaac's syndrome, neuromyotonia, kuru, opsoclonus myoclonus, Kinsboume syndrome, spinal muscular atrophy, Werdnig-Hoffman disease, Kugelberg-Welander disease, transmissible spongiform encephalopathies, fatal familial insomnia, Lambert-Eaton myasthenic syndrome, Leigh's disease, locked-in syndrome, Lou Gehrig's disease, lupus, systemic lupus erythematosus, Machado-Joseph disease, Melkersson-Rosenthal syndrome, Miller Fisher syndrome, mitochondrial myopathies, motor neuron disease, multifocal motor neuropathy, multiple sclerosis, multiple system atrophy, muscular dystrophy, myasthenia gravis, neurofibromatotis, von Recklinghausen's disease, neurological complications of Lyme's disease, thyrotoxic myopathy, tabes dorsalis, progressive locomrotor ataxia, prion diseases, primary lateral sclerosis, acute demyelinating neuropathy, acute disseminated encephalomyelitis, acute necrotizing hemorrhagic leukoencephalitis, metachromic leukodystrophy, adrenoleukodystrophy, adrenomyeloneuropathy, spinocerebellar degenerations, mitochoncrial encephalomyopathies, Pelizaeus-Merzbacher disease, creatine transporter defect, and Duchenne muscular dystrophy. In one embodiment, the neurological disorder is Huntington's disease, Parkinson's disease, amyotrophic lateral sclerosis or creatine transporter defect.

In one embodiment, the invention pertains, at least in part, to methods of treating creatine transporter defect in a subject in which an effective amount of a creatine composition of formula (I) is administered to the subject, wherein the creatine composition of formula (I) is comprised of between about a 4:1 ratio of z:y (creatine to ascorbylphosphate) to about an 8:1 ratio z:y (creatine:ascorbyl phosphate). In another embodiment, the invention pertains, at least in part to methods of treating creatine transporter defect in a subject in which an effective amount of a creatine composition of formula (I) is administered to the subject, wherein the creatine composition is comprised of between about a 2:1 ratio of z:y (creatine:ascorbyl phosphate) to about an 8:1 ratio of z:y.

The term “creatine transporter defect” includes a disorder characterized by an inborn error creatine synthesis or of the creatine transporter or other aberrant creatine transport function in the brain. The aberrant creatine transport function in the brain may cause the subject to suffer from a low concentration of creatine in the brain of a subject suffering from creatine transporter dysfunction. In this disorder, impaired energy metabolism is believed to be associated with impaired learning dysfunction and cognitive function. It was found that treatments of similar neurological or cognitive dysfunctions do not tend to target improving metabolism and/or energy metabolism of the brain, neural cells, or glial cells.

In yet another embodiment, the invention pertains, at least in part, to a method of treating amyotrophic lateral sclerosis in a subject comprising administering to the subject an effective amount of a creatine composition of formula (I), wherein the creatine composition of formula (I) is comprised of between about a 2:1 ratio of creatine to ligand and about an 8:1 ratio of z:y. In one embodiment, the creatine composition of formula (I) is comprised of about a 2:1 ratio of z:y.

In yet another embodiment, the invention pertains, at least in part, to a method of treating amyotrophic lateral sclerosis in a human by administering to the human a composition comprising a creatine composition of formula (I) (e.g., creatine ascorbyl phosphate) and dextrose.

In another embodiment, the present invention pertains, at least in part, to methods of treating a neurological disorder in a subject in which an effective amount of a creatine composition of formula (I) in combination with a second agent, e.g., a neuroprotective agent, an anti-inflammatory compound, a COX-2 inhibitor, etc., is administered to the subject. In one particular embodiment, the creatine composition of formula (I) is administered with dextrose.

In another embodiment, the present invention pertains, at least in part, to methods of beating a neurological disorder in a subject in which an effective amount of a creatine composition of formula (I) in combination with a second agent is administered to the subject.

The term “second agent” includes anti-inflammatory compounds, COX-2 inhibitors, neuroprotective agents, tetracyclines, botanical additives, and other compounds which may be advantageously administered with the creatine composition of formula (I). Other second agents also include low molecular proteins and polypeptides (e.g., polypeptides with a mass less than about 2000, less than about 1000, less than about 500, or less than about 250 daltons).

Neuroprotective agents include: approved drugs for the treatment or prevention of neurodegenerative diseases such as Riluzole, Cognex, Aricept, Sinmet; Sinmet CR, Permax, Parlodel, Elepryl, Symmetrel, Artane); glutamate excitotoxicity inhibitors (such as glutamate uptake and biosynthesis modulation with compounds like gabapentin and Riluzole); growth factors like CNTF, BDNF, IGF-1; nitric oxide synthase inhibitors; cyclo-oxygenase inhibitors such as aspirin; ICE inhibitors; Neuroimmunophilins; N-acetylcysteine and procysteine; antioxidants (such as pyruvate and lutein), energy enhancers (such as ribose and vincopocetine), vitamins and cofactors (such as spin traps, CoQ₁₀, carnitine, nicotinamide, Vitamin E or D) lipoic acid, vinpocetine, other fatty acids (such as docosahexanoic acid (DHA), eicosopentenoic acid (EPA), and gamma linolenic acid (GLA)), various herbal extracts (such as rosemary and black caraway), and berry oils and meals (such as bilberry, elderberry, english hawthorn berry, blackberry, blueberry, red and black raspberries).

The term “anti-inflammatory compound” refers compounds that treat, prevent or ameliorate inflammation in a subject. The anti-inflammatory compounds of the present invention include, for example, members of the tetracycline family, opiate agonists, lipoxygenase inhibitors, cyclooxygenase inhibitors (e.g., cyclooxygenase-1 (COX-1) selective inhibitors, cyclooxygenase-2 (COX-2) selective inhibitors and non-selective cyclooxygenase inhibitors), interleukin receptor antagonists, NMDA receptor antagonists, inhibitors of nitric oxide or inhibitors of the synthesis of nitric oxide, non-steroidal anti-inflammatory agents, steroidal anti-inflammatory compounds or cytokine-suppressing anti-inflammatory agents.

In one embodiment, the anti-inflammatory compound may be a member of the tetracycline family. The language “member of the tetracycline family” includes many compounds with a similar ring structure to tetracycline. Examples of tetracycline compounds include: oxytetracycline, demeclocycline, methacycline, minocycline, sancycline, chelocardin, rolitetracycline, lymecycline, apicycline; clomocycline, guamecycline, meglucycline, mepylcycline, penimepicycline, pipacycline, etamocycline, penimocycline, etc. Other derivatives and analogues comprising a similar four ring structure are also included (See Rogalski, “Chemical Modifications of Tetacyclines,” the entire contents of which are hereby incorporated herein by reference). Table 1 depicts tetracycline and several known members of the tetracycline family.

TABLE 1

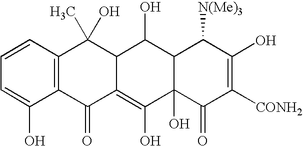

Oxytetracycline

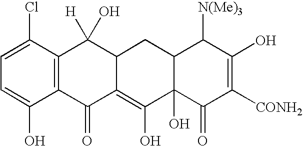

Demeclocycline

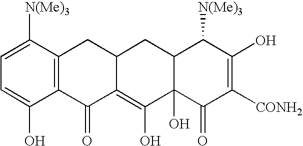

Minocycline

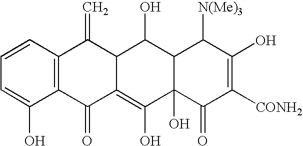

Methacycline

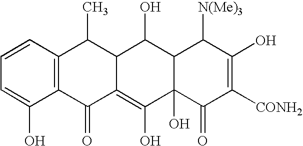

Doxycycline

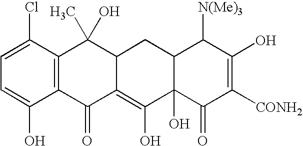

Chlortetracycline

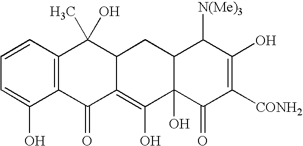

Tetracycline

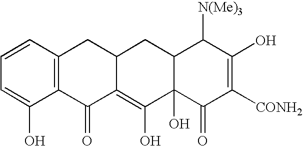

Sancycline

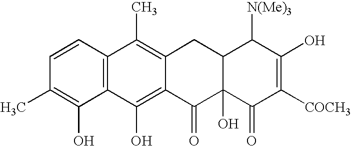

Chelocardin

In one embodiment, the member of the tetracycline compound is selected from the group consisting of oxytetracycline, demeclocycline, minocycline, methacycline, doxycycline, chlortetracycline, tetracycline, sancycline, chelocardin and pharmaceutically acceptable derivatives thereof. In one embodiment, the member of the tetracycline family is minocycline. The dosage of the member of the tetracycline family may be between about 50 and 500 mg per day. In one embodiment, the dosage is 400 mg. In another embodiment, the dosage is about 100 mg per day.

In another embodiment, the anti-inflammatory may be a cyclooxygenase-2 (COX-2) selective inhibitor. The language “cyclooxygenase-2 (COX-2) selective inhibitor” refers to compounds that selectively inhibit the cyclooxygenase-2 enzyme over the cyclooxygenase-1 enzyme. Suitable COX-2 inhibitors include, for example, 2-(4-ethoxy-phenyl)-3-(4-methanesulfonyl-phenyl)-pyrazolo[1,5-b]pyridazine, CDC-501, celecoxib, COX-189, CS-179, CS-502, D-1367, 4-(2-oxo-3-phenyl-2,3-dihydrooxazol-4-yl)benzenesulfonamide, darbufelone, DFP, DRF-4367, etodolac, flosulide, JTE-522 (4-(4-cyclohexyl-2-methyl-5-oxazolyl)-2-fluorobenzenesulfonamide), L-745337, L-748731, L-748780, L-768277, L-776967, L-783003, L-791456, L-804600, L-748706, meloxicam, MK663 (etoricoxib), nimesulide, NS-398, parecoxib, 1-methylsulfonyl-4-(1,1-dimethyl-4-(4-fluorophenyl)cyclopenta-2,4-dien-3-yl)benzene, DuP-697, L-761066, 4-(1,5-dihydro-6-fluoro-7-methoxy-3-(trifluoromethyl)-(2)-benzothiopyrano-(4,3-c)pyrazol-1-yl)benzenesulfonamide, 4,4-dimethyl-2-phenyl-3-(4-methylsulfonyl)phenyl)cyclobutenone, 4-amino-N-(4-(2-fluoro-5-trifluoromethyl)-thiazol-2-yl)benzene sulfonamide, meloxicam, 1-(7-tert-butyl-2,3-dihydro-3,3-dimethyl-5-benzo-furanyl)-4-cyclopropyl-butan-1-one, rofecoxib, RS-113472, RWJ-63556, RS-57067, S-2474, S-33516, SC-299, SC-5755, SC-57666, SC-58125, lumiracoxib valdecoxib, parecoxib sodium, APHS, UR-8877, UR-8813, UR-8880 and pharmaceutically acceptable derivatives thereof. In one embodiment, the COX-2 selective inhibitor is celecoxib. In another embodiment, the dosage of the COX-2 selective inhibitor is between about 100 and 500 mg per day. In a further embodiment, the dosage of the COX-2 selective inhibitor is about 400 mg per day.

In certain embodiments, the second agent is a botanical additive. As used herein, the term “botanical additive” indicates any compound obtained from a natural source, including plants, bacteria and yeast, which has a medicinal or otherwise beneficial effect when topically applied to the skin or when otherwise administered to a subject. Examples of botanical additives include, without limitation, oil of Melaleuca spp. (tea tree oil), oil of Lavandula angustifolia, Carica papaya extract, Echinacea angustifolia extract, Mimosa tenuiflora extract, Hydrocotyl (centella,) asiatica extract, gingko biloba extract, Matricaria chamomila (chamomile oil) extract, Hypericum perforatum extract, Aloe barbedensis extract, and the like. The botanical sources for “botanical additives” may also include, but are not limited to the following: Aloe Vera, (e.g., Aloe Barbedensis); Arnica, (e.g., Arnica Montana); Bladderwrack (seaweed), (e.g., Fucus Vesciculosis); Birch, (e.g., Betula Alba) (Pendula); Chamomile, (e.g., Matricaria Chamomila, Chamomila Recutita); Marsh Mallow, (e.g., Althea Officinalis); Meadow Sweet, (e.g., Spirea Ulmaria) (Filipendula); Mint/Lemon Balm, (e.g., Melissa Officinalis); Mimosa, (e.g., Mimosa Tenuiflora); Myrrh Tincture, (e.g., Commiphor Myrrha); Neem, (e.g., Melia Azadirachta); Nettle (stinging), (e.g., Urtica Dioica); Papaya, (e.g., Carica Papaya); Propolis (bee glue), (e.g., Propolis Cera); Raspberry, (e.g., Rubis Idaeus); Red Poppy, (e.g., Papaver Rhoeas); Rose Hip (dog rose), (e.g., Rosa Carima); Rosemary, (e.g., Rosemarinus Officinalis); Sage, (e.g., Salvia Officinalis); St. Johns Wort, (e.g., Hypericum Perforatum); Strawberry, (e.g., Fragaria Vesca); Thea Sinensis (green tea), (e.g., Camelia Sinensis); Walnut, (e.g., Juglans Regia); Witchhazel (dist/extr), (e.g., Hamamelis Virginiana); Yarrow, (e.g., Achillea Millefoliwn); Wild Yam, (e.g., Dioscorea Villosa); Hawthorn, (e.g., Crataegus Monogina/Oxyantha); Herma (black/rod), (e.g., Lawsoma Ehemus); Hops, (e.g., Humulus Lupulus); Horse Chestnut, (e.g., Aesculus Hippocastanum); Horse Tail, (e.g., Equisitum Arvense); Ivy, (e.g., Hedera Helix); Linden/Lime Tree Blossoms, (e.g., Tilia Argentea Cordata); Madder, (e.g., Rubia Tinctorum); Marigold, (e.g., Calendula Officinalis, Centella Asiatica, Centella Asiatica Urban, Hydrocotyl Asiatica); Carrot (roots), (e.g., Daucus Carota); Comfrey (Allantoine), (e.g., Symphytum Officinale); Coneflower (Echinacea), propolis (e.g., Echinacea Angustifolia); Cucumber, (e.g., Cucumis Sativus, Frucus Cucumis); Fenugreek, (e.g., Trigonella Foenum Greacum); Gingko, (e.g., Gingko Biloba); Ginseng, (e.g., Panax Ginseng); Great Burdock, (e.g., Radix Bardanea/Arctium Lappa); Tea Tree Oil, (e.g., Oil of Melaleuca Alternifolia); Colts Foot, (e.g., Tussilago Farfara); Clover, arbutui (e.g., Trifolium Pratense); Speedwell, (e.g., Veronica Officinalis). A particularly preferred biological additive is tea tree oil.

The language “in combination with” a second agent includes co-administration of the creatine composition of formula (I) and with a second agent, administration of the creatine composition of formula (I) first, followed by administration of the second agent, and administration the second agent first, followed by administration of the creatine composition of formula (I). The creatine composition of formula (I) can be administered substantially at the same time as the second agent or at substantially different times. Optimal administration rates for a given protocol of administration of the creatine composition of formula (I) and/or the second agent can be readily ascertained by those skilled in the art using conventional dosage determination tests conducted with regard to the specific compounds being utilized, the particular compositions formulated, the mode of application, the particular site of administration and the like.

2. Skin Disorders

In another embodiment, the creatine responsive state is a skin disorder. Examples of skin disorder include, but are not limited to, aging, damage resulting from sun radiation, stress, uneven pigmentation, psoriasis, fatigue and/or damage associated with free radicals. In another embodiment, the subject is at risk of suffering from a skin disorder. In a further embodiment, the subject is afflicted with skin wrinkles. The language “treating for skin disorders” includes both prevention of disorders, amelioration and/or arrest of the disorder process. The language also includes any amelioration or arrest of any symptoms associated with the disorder process (e.g., wrinkles), For example, treating wrinkles may include preventing, retarding, arresting, or reversing the process of wrinkle formation in skin, e.g., mammalian skin, preferably, human skin.

The term “aging” includes processes where there is oxidative damage, energy depletion or mitochondrial dysfunction where onset, amelioration, arrest, or elimination is effectuated by the creatine compounds described herein. Symptoms of aging include, but are not limited to, wrinkles, loss of elasticity of the skin and uneven pigmentation of the skin.

The term “associated with free radicals” includes any disorders or damaged to the skin resulting from, directly or indirectly from free radicals. The free radicals may be initiated by, for example, sun radiation (e.g., UV radiation) or pollution.

In one embodiment, the invention pertains, at least at part, a method of treating a skin disorder in which an effective amount of a creatine composition of formula (I) is administered to a subject such that the skin disorder in said subject is treated. In one embodiment, the treatment of the skin disorder reduces or eliminates at least one preexisting symptom of the skin disorder. The preexisting symptom may include, for example, skin wrinkles or a loss of skin elasticity. In another embodiment, the treatment of the skin disorder comprises the prevention of the skin disorder.

In yet another embodiment the method of treating a skin disorder further comprises co-administering to a subject an effective amount of a creatine composition of formula (I) and an effective amount of a skin preserving agent. Examples of skin preserving agents include antioxidants, such as ascorbic acid, vitamins, coenzyme Q10 (CoQ10) and its derivatives, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Preferred anti-oxidants include, CoQ10 and vitamin E. Other examples of skin preserving agents include energy-enhancing agents (e.g. ATP, nicotinamide or pyruvate), vitamins (e.g., E, C, B5, B6, and B9) and vitamin precursors.

The term “energy enhancing agents” also includes stimulants of mitochondrial function or ATP production elsewhere in the cell. Examples include intermediates such as, for example, pyruvate, nicotinamide and CoQ10.

The term “subject” is intended to include living organisms susceptible to having creatine responsive states (e.g., mammals). Examples of subjects include humans, dogs, cats, horses, cows, goats, rats and mice. The term “subject” also includes include transgenic species. In one embodiment, the subject is a human.

### Pharmaceutical Compositions

In one embodiment, the invention pertains, at least in part, to a pharmaceutical composition comprising an effective amount of a creatine composition of formula (I) and an acceptable carrier, wherein said effective amount is effective for the treatment of a creatine responsive state. In another embodiment, the invention pertains, at least in part, to a pharmaceutical composition comprising an effective amount of a creatine composition of formula (I) in combination with a second agent, e.g., a neuroprotective agent, a COX-2 inhibitor, an anti-inflammatory compound, a neuroprotective agent, etc., and an acceptable carrier, wherein said effective amount is effective for the treatment of a creatine responsive state. In one embodiment, the acceptable carrier is suitable for oral or topical administration.

The phrase “acceptable carrier” includes a pharmaceutically or cosmetically acceptable material, composition or vehicle, such as a liquid or solid filler, diluent, excipient, solvent or encapsulating material, involved in carrying or transporting the creatine composition of formula (I) and/or a second agent within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the subject. Some examples of materials which can serve as pharmaceutically or cosmetically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical or cosmetic formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically and cosmetically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The pharmaceutical compositions of the present invention may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Those skilled in the art related to the present invention will be better able to determine an appropriate dosage and overall dosage regime when taking a number of factors into consideration. For example, the size, weight and condition of the patient must be considered as must be the responsiveness of the patient and their disorder to the particular therapy. In one embodiment, the dosage is from 0.001 μg to 100 g and may be administered once or several times daily, weekly, monthly or yearly, or even every 2 to 20 years. In one embodiment, a suitable dose of a compound of the invention will be that amount of the compound which is the lowest dose effective to produce a therapeutic effect. In another embodiment, a suitable dose of a compound of the invention will be is an effective daily dose, which includes the lowest daily dose effective to produce a therapeutic effect. The effective daily dose of the active compound may be administered as two, three, four, five, six or more sub-doses administered separately at appropriate intervals throughout the day, optionally, in unit dosage forms.

The language “effective amount” of the compound is that amount necessary or sufficient to treat, prevent or ameliorate a creatine responsive state in a subject. The effective amount can vary depending on such factors as the size and weight of the subject, the type of illness, etc. One of ordinary skill in the art would be able to study the aforementioned factors and make the determination regarding the effective amount of the creatine composition of formula (I) and/or the second agent without undue experimentation.

In one embodiment, the effective amount of the creatine composition of formula (I) is a dosage of the creatine composition of formula (I). The term “dosage of creatine composition of formula (I)” refers to a specified quantity of the creatine composition of formula (I). In one embodiment, the dosage of the creatine composition of formula (I) is between about 5 grams and about 50 grams. The term “about,” as used with reference to a dosage of the creatine composition of formula (I) and/or the second agent refers tot 0.5 grams of the creatine composition of formula (I) and/second agent. In another embodiment, the dosage of the creatine composition of formula (I) is about 5 grams, about 6 grams, about 7 grams, about 8 grams, about 9, grams, about 10 grams, about 11 grams, about 12 grams, about 13 grams, about 14 grams, about 15 grams, about 16, grams, about 17 grams, about 18 grams, about 19 grams, about 20 grams, about 21 grams, about 22 grams, about 23 grams, about 24 grams, about 25 grams, about 26 grams, about 27 grams, about 28 grams, about 29 grams, about 30 grams, about 31 grams, about 32 grains, about 33 grams, about 34 grams, about 35 grams, about 36 grams, about 37 grams, about 38 grams, about 39 grams, about 40 grams, about 41 grams, about 42 grams, about 43 grams, about 44 grams, about 45 grams, about 46 grams, about 47 grams, about 48 grams, about 49 grams or about 50 grams or greater. In another embodiment, the dosage of the creatine composition of formula (I) is a therapeutically effective amount.

In one embodiment, the dosage of the creatine composition of formula (I) for the treatment of a creatine-responsive state is between about 5 grams and 50 grams, or, alternatively, about 40 grams. In another embodiment, the creatine composition of formula (I) in said dosage is comprised of between about a 1:1 ratio of z:y, alternatively, the ratio may be about 3:1, z:y.

1. Oral, Nasal, Transdermal, Buccal, Sublingual and/or Parenteral Administration

In one embodiment, formulations of the invention include those suitable for oral, nasal, transdermal, buccal, sublingual and/or parenteral administration. The formulations may conveniently be presented in unit dosage form and may be prepared by any methods well known in the art of pharmacy. Methods of preparing these formulations or compositions include the step of bringing into association the creatine composition of formula (I) and/or a second agent, with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Formulations of the invention suitable for oral administration may be in the form of capsules, cachets, pills, wafers, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary or paste.

In solid dosage forms of the invention for oral administration (capsules, tablets, wafers, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the creatine composition of formula (I) and/or second agents include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for transdermal administration of compounds of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the creatine composition of formula (I) and/or a second agent, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the creatine composition of formula (I) and/or a second agent to the body. Such dosage forms can be made by dissolving or dispersing the compound in the proper medium, Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise the creatine composition of formula (I) and/or a second agent in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound, it is desirable to slow the absorption of the compound from subcutaneous or intramuscular injection. This may be accomplished by the use of a liquid suspension of crystalline or amorphous material having poor water solubility. The rate of absorption of the compound then depends upon its rate of dissolution which, in turn, may depend upon crystal size and crystalline form. Alternatively, delayed absorption of a parenterally-administered compound form is accomplished by dissolving or suspending the compound in an oil vehicle.

Injectable depot forms are made by forming microencapsule matrices of the compounds of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The creatine composition of formula (I) and/or the second agent may be administered to humans and other animals for therapy by any suitable route of administration, including orally, nasally, as by, for example, a spray, parenterally, intracistemally and topically, as by powders, ointments or drops, including buccally and sublingually.

2. Topical Administration

In one embodiment, the acceptable carrier is suitable for topical administration The creatine composition of formula (I) and/or the second agent may be suitable for administration as a lotion, cream, mousse, aerosol, gel, emulsion, solution, ointment, or medicated pad.

The topical pharmaceutical compositions of the present invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active compound, and possesses acceptable safety properties (e.g., irritation and sensitization characteristics). Water is a typical aqueous solvent. Examples of suitable organic solvents include: propylene glycol, butylene glycol, polyethylene glycol (200-600), polypropylene glycol (425-2025), glycerol, 1,2,4-butanetriol, sorbitol esters, 1,2,-6-hexanetriol, ethanol, isopropanol, butanediol, and mixtures thereof. Preferably, these solutions contain from about 0.01% to about 50% of the active compound, more preferably from about 0.1% to about 20%; and from about 1% to about 80% of an acceptable aqueous or organic solvent, more preferably from about 1% to about 40%.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

Topical pharmaceutical compositions of the present invention may be formulated as a solution comprising an emollient. An example of a composition formulated in this way would be a sunscreen-containing product. Preferably, such compositions contain from about 0.1% to about 50% of the active compound and from about 2% to about 50% of a topical pharmaceutically-acceptable emollient.

As used herein, “emollients” refer to materials used for the prevention or relief of dryness, as well as for the protection of the skin. A wide variety of suitable emollients is known and may be used herein. Sagarin, Cosmetics, Science and Technology, 2nd Edition, Vol. 1, pp. 32-43 (1972), incorporated herein by reference, contains numerous examples of suitable materials.

A lotion can be made from a solution carrier system. Lotions preferably comprise from about 0.1% to about 20%, more preferably from about 1% to about 5%, of the active compound; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.1% to about 20%, more preferably from about 1% to about 5%, of the active compound; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of thickening agents useful herein can be found in Segarin, Cosmetics, Science and Technology, 2nd Edition, Vol. 1, pp. 72-73 (1972).

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560, issued Aug. 28, 1973, Dickert et al; U.S. Pat. No. 4,421,769, issued Dec. 20, 1983, Dixon et al.; and McCutcheon's Detergents and Emulsifiers, North American Edition, pages 317-324 (1986); the disclosures of which are incorporated herein by reference. Preferred emulsifiers are anionic or nonionic, although the other types may also be used.

Lotions and creams can be formulated as emulsions as well as solutions. Preferably such lotions comprise from about 0.1% to about 20%, more preferably from about 1% to about 5%, of the active compound; from about 1% to about 20%, preferably from about 5% to about 10%, of an emollient; from about 25% to about 75%, preferably from about 45% to about 95%, water, and from about 0.1% to about 10%, preferably from about 0.5% to about 5%, of an emulsifier. Such creams would preferably comprise from about 0.1% to about 20%, more preferably from about 1% to about 5%, of the active compound; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type, as disclosed in U.S. Pat. No. 4,254,105, Fakuda et al., issued Mar. 3, 1981, incorporated herein by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. Pat. No. 4,960,764, Figueroa, issued Oct. 2, 1990, are also useful in the present invention. Preferably, this triple emulsion carrier system can be combined with from about 0.1% to about 20%, more preferably from about 1% to about 5%, of the active compound to yield the topical pharmaceutical composition of the present invention.

Another emulsion carrier system useful in the topical pharmaceutical compositions of the present invention is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is preferably combined with from about 1% to about 5% of the active compound.

If the topical pharmaceutical compositions of the present invention are formulated as a gel or a cosmetic stick, a suitable amount of a thickening agent, as disclosed supra, is added to a cream or lotion formulation.

The topical pharmaceutical compositions of the present invention may also be formulated as makeup products such as foundations.

The topical pharmaceutical compositions of the present invention may also be formulated as medicated pads. Suitable examples of these pads are fully disclosed in U.S. Pat. Nos. 4,891,227 and 4,891,228, to Thaman et al., both issued Jan. 2, 1990 the disclosures of which are incorporated herein.

The topical pharmaceutical compositions of the present invention may contain, in addition to the aforementioned components, a wide variety of additional oil-soluble materials and/or water-soluble materials conventionally used in topical compositions, at their art-established levels.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments and perfumes.

The topical pharmaceutical compositions of the present invention may also include a safe and effective amount of a penetration enhancing agent. A preferred amount of penetration enhancing agent is from about 1% to about 5% of the composition. Another useful penetration enhancer for the present invention is the non-ionic polymer under the CTFA designation: polyacrylamide and isoparrafin and laureth-7, available as Sepigel from Seppic Corporation. Also useful is polyquaternium-32 and mineral oil known as SalCare SC92 available from Allied Colloids, Suffolk, Va. This is a class of cationic polymers which are generally described in U.S. Pat. No. 4,628,078 to Glover et al. issued Dec. 9, 1986 and U.S. Pat. No. 4,599,379 to Flesher et al. issued Jul. 8, 1986 both of which are incorporated by reference herein.

Examples of useful penetration enhancers, among others, are disclosed in U.S. Pat. Nos. 4,537,776, Cooper, issued Aug. 27, 1985; 4,552,872, Cooper et al., issued Nov. 12, 1985; 4,557,934, Cooper, issued Dec. 10, 1985; 4,130,667, Smith, issued Dec. 19, 1978; 3,989,816, Rhaadhyaksha, issued Nov. 2, 1976; 4,017,641, DiGiulio, issued Apr. 12, 1977; and European Patent Application 0043738, Cooper at al., published Jan. 13, 1982.

Other conventional ski care product additives may also be included in die compositions of the present invention. For example, collagen, hyaluronic acid, elastin, hydrolysates, primrose oil, jojoba oil, epidermal growth factor, soybean saponins, low molecular weight peptides and proteins, botanical additives, mucopolysaccharides, and mixtures thereof may be used.

Further examples of additives include, but are, not limited to, aloe, echinacea, green tea extract, ginseng, gingko biloba, whole grape extract, amino acids (e.g., proline and glycine), chamomile, feverfew, glucosamine, lipoic acid, milk thistle, chromium, veronica beccabunga, veronica officialis, nettle, cranberry see oil and mixtures thereof.

Various vitamins may also be included in the compositions of the present invention. For example, Vitamin A, ascorbic acid, Vitamin B, biotin, panthothenic acid, Vitamin D, Vitamin E and mixtures thereof and derivatives thereof are contemplated.

Also contemplated are skin cleaning compositions comprising both active compounds of the present invention and a cosmetically-acceptable surfactant. The term. “cosmetically-acceptable surfactant” refers to a surfactant which is not only an effective skin cleanser, but also can be used without undue toxicity, irritation, allergic response, and the like. Furthermore, the surfactant must be capable of being commingled with the active compound in a manner such that there is no interaction which would substantially reduce the efficacy of the composition for regulating skin damage, e.g., wrinkles.

The skin cleaning compositions of the present invention preferably contain from about 0.1% to about 20%, preferably from about 1% to about 5%, of the creatine composition of formula (I) and from about 1% to about 90%, more preferably from about 1% to about 10%, of a cosmetically-acceptable surfactant.

The physical form of the skin cleansing compositions is not critical. The compositions can be, for example, formulated as toilet bars, liquids, pastes, mousses, or pads.

The surfactant component of the compositions of the present invention are selected from anionic, nonionic, zwitterionic, amphoteric and ampholytic surfactants, as well as mixtures of these surfactants. Such surfactants are well-known to those skilled in the detergency art.

The cleaning compositions of the present invention can optionally contain, at their art-established levels, materials which are conventionally used in skin cleansing compositions.

Sunblocks and sunscreens incorporating creatine compounds are also contemplated. The term “Sun block” or “sun screen” includes compositions which block UV light. Examples of sunblocks include, for example, zinc oxide and titanium dioxide.

Sun radiation is one cause major cause of skin damage, e.g., wrinkles. Thus, for purposes of wrinkle treatment or prevention, the combination of creatine compounds with a UVA and/or UVB sunscreen would be advantageous. The inclusion of sunscreens in compositions of the present invention will provide immediate protection against acute UV damage. Thus, the sunscreen will prevent flintier skin damage caused by UV radiation, while the compounds of the invention regulates existing skin damage.

A wide variety of conventional sunscreening agents are suitable for use in combination with the active compound. Segarin, et al., at Chapter VIII, pages 189 et seq., of Cosmetics Science and Technology, disclose numerous suitable agents. Specific suitable sunscreening agents include, for example: p-aminobenzoic acid, its salts and its derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters), cinnamic acid derivatives (methyl and benzyl esters, alpha-phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4 isopropyl-di-benzoylmethane; butylmethoxydibenzoylmethane; etocrylene; and 4-isopropyl-1-benzoylmethane.

Preferred sunscreens useful in the compositions of the present invention are 2-ethylhexyl-p-methoxycinnamate, butylmethoxydibenzoylmethane, 2-hydroxy-4-methoxybenzophenone, octyldimethyl-p-aminobenzoic acid and mixtures thereof.

A safe and effective amount of sunscreen may be used in the compositions of the present invention. The sunscreening agent must be compatible with the active compound Generally the composition may comprise from about 1% to about 20%, preferably from about 2% to about 10%, of a sunscreening agent. Exact amounts will vary depending upon the sunscreen chosen and the desired Sun Protection Factor (SPF).

Also included are sunscreens such as those disclosed in Sabatelli, U.S. patent application Ser. No. 054,085 (filed Jun. 2, 1987) and Sabatelli et al, U.S. patent application Ser. No 054,046 (filed Jun. 2, 1987). The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range.

Additional agents may also be added to any of the compositions of the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987, which is incorporated herein by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

The entire contents of all references, patents, and patent applications cited herein are expressly incorporated by reference.

1. A creatine composition of the formula (I): 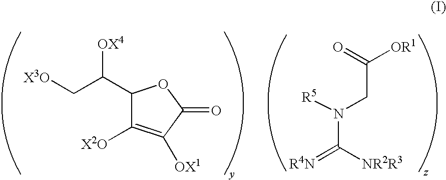
wherein:
X¹, X², X³, and X⁴ are each independently hydrogen, phosphate, diphosphate, triphosphate, sulfate, or carboxylate, provided that at least one of X¹, X², X³, and X⁴ is not hydrogen;
R¹ is hydrogen, phosphate, diphosphate, triphosphate, sulfate, or carboxylate;
R², R³, R⁴ and R⁵ are each independently alkyl or hydrogen; and
y and z are each independently selected integers, or a pharmaceutically acceptable salt or tautomer thereof.

2. The creatine composition of claim 1, wherein each of R¹, R², R³, and R⁴ are hydrogen and R⁵ is methyl.

3. (canceled)

4. The creatine composition of claim 1, wherein z is greater than or equal to y.

5. (canceled)

6. The creatine composition of claim 5, wherein X¹ is phosphate and X², X³, and X⁴ are each hydrogen.

7. (canceled)

8. The creatine composition of claim 1, wherein said compound is:

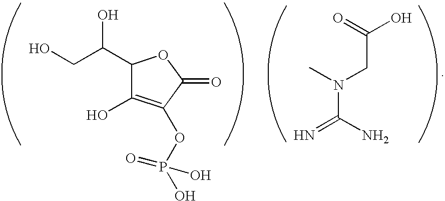

9. The composition of claim 1, wherein said composition further comprises a bivalent metal selected from the group consisting of magnesium, calcium, iron, zinc, selenium or chromium.

10. (canceled)

11. The composition of claim 1, wherein said composition is creatine magnesium ascorbyl phosphate.

12. A pharmaceutical composition comprising a pharmaceutically acceptable carrier and a creatine composition of the formula (I): 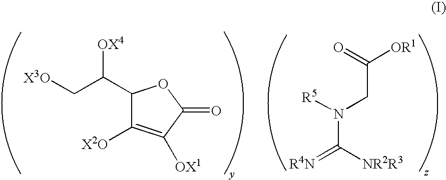
wherein:
X¹, X², X³, and X⁴ are each independently hydrogen, phosphate, diphosphate, triphosphate, sulfate, or carboxylate, provided that at least one of X¹, X², X³, and X⁴ is not hydrogen;
R¹ is hydrogen, phosphate, diphosphate, triphosphate, sulfate, or carboxylate;
R², R³, R⁴ and R⁵ are each independently alkyl or hydrogen; and
y and z are each independently selected integers, or a pharmaceutically acceptable salt or tautomer thereof.

13. The pharmaceutical composition of claim 12, wherein said pharmaceutically acceptable carrier is dextrose.

14. The pharmaceutical composition of claim 12, wherein said pharmaceutically acceptable carrier is suitable for oral or topical administration.

15. (canceled)

16. The pharmaceutical composition of claim 12, wherein said pharmaceutical composition comprises an effective amount of the creatine composition to treat a creatine responsive state.

17. The pharmaceutical composition of claim 16, wherein said creatine responsive state is a neurological disorder or a skin disorder.

18. (canceled)

19. (canceled)

20. The pharmaceutical composition of claim 12, wherein acceptable carrier is suitable for administration as a lotion, cream, mousse, aerosol, gel, emulsion, solution, ointment, or medicated pad.

21. (canceled)

22. (canceled)

23. The pharmaceutical composition of claim 12, wherein said composition of formula (I) is creatine magnesium ascorbyl phosphate.

24. A method of treating a creatine responsive state in a subject comprising administering to said subject a composition comprising an effective amount of a creatine composition of formula (I), such that the creatine responsive state in said subject is treated, wherein said creatine composition of formula (I) is: 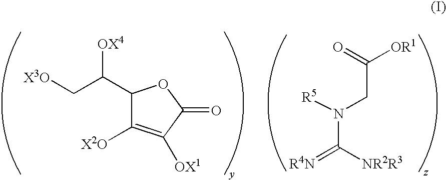
wherein:
X¹, X², X³, and X⁴ are each independently hydrogen, phosphate, sulfate, or carboxylate, provided that at least one of X¹, X², X³, and X⁴ is not hydrogen;
R¹ is hydrogen, phosphate, sulfate, or carboxylate;
R², R³, R⁴ and R⁵ are each independently alkyl or hydrogen; and
y and z are each independently selected integers, or a pharmaceutically acceptable salt or tautomer thereof.

25. The method of claim 24, wherein each of R¹, R², R³, and R⁴ are hydrogen and R⁵ is methyl.

26. The method of claim 24, wherein z is greater than or equal to y.

27. (canceled)

28. The method of claim 24, wherein X¹ is phosphate and wherein X², X³, and X⁴ are each hydrogen.

29.-31. (canceled)

32. The method of claim 24, wherein said composition of formula (I) is creatine magnesium ascorbyl phosphate.

33. The method of claim 24, wherein said creatine responsive state is a neurological disorder or a skin disorder.

34.-37. (canceled)

38. The method of claim 24, wherein said subject is human.

39. The method of claim 24, wherein said subject is at risk of suffering from a neurological disorder or a skin disorder.

40. (canceled)

41. (canceled)

42. The method of claim 24, further comprising administering a second agent.

43. The method of claim 42, wherein said second agent is an anti-inflammatory compound or botanical additive.

44.-63. (canceled)
